(19) United States
(12) Patent Application Publication
(10) Pub. No.: US 2015/0066094 A1
(43) Pub. Date: Mar. 5, 2015

(54) ORTHOPEDIC IMPLANT IN THE FORM OF A PLATE TO BE FIXED BETWEEN TWO BONE PARTS

(71) Applicant: Memometal Technologies, Bruz (FR)

(72) Inventors: Bernard Prandi, Rennes (FR); Marino Delmi, Conches (CH); J. Chris Coetzee, Eagan, MN (US); Lowell Well, Lake Forest, IL (US); Keith Wapner, Philadelphia, PA (US); Christopher P. Chiodo, Jamaica Plain, MA (US); Michael Coughlin, Boise, ID (US); Yves Tourne, Gieres (FR); Christophe Richard Walker, Loirral (GB); Donald R. Bohay, Ada, MI (US); John G. Anderson, Lowell, MI (US)

(21) Appl. No.: 14/534,774

(22) Filed: Nov. 6, 2014

Related U.S. Application Data
(63) Continuation of application No. 12/918,116, filed on Nov. 1, 2010, now abandoned.

(30) Foreign Application Priority Data
Oct. 2, 2008 (FR) 0856693

(51) Int. Cl. A61B 17/80; A61B 17/86

(52) U.S. Cl. CPC A61B 17/8057; A61B 17/86
USPC 606/291

(57) ABSTRACT

A plate is disclosed for compressing together first and second bone parts. The plate includes screw holes adapted to receive screws for insertion into first and second bone parts, respectively, at least one of the screw holes being a compression slot with formations configured to ensure, when a respective screw is seated within the slot, an adjustable compression force adapted to bring the first and second bone parts closer together. The plate also comprises a circular guide pin hole for insertion of a pin into one of the first and second bone parts, and another guide pin hole, in the form of an elongate slot, for insertion of a pin into the other of the first and second bone parts. The plate may also include a curvature adapted to cause at least a pair of screws inserted into screw holes of the plate to converge.

### CROSS-REFERENCE TO RELATED APPLICATIONS

The present application is a continuation of U.S. patent application Ser. No. 12/918,116, filed Nov. 1, 2010, which is a national phase entry under 35 U.S.C. §371 of International Application No. PCT/FR2009/051878, filed Oct. 2, 2009, which claims priority from French Patent Application 0856693, itself filed Oct. 2, 2008, all of which are herby incorporated herein by reference.

### BACKGROUND OF THE INVENTION

The invention relates to the technical field of orthopedic implants.

More particularly, the invention relates to a plate for arthrodesis or osteosynthesis adapted to be fixed between two bone parts.

In a manner known to one having ordinary skill in the art, this type of plate generally has holes for anchor screws, allowing arthrodesis between two bones or an osteosynthesis between two bone fragments. This is, for example, the case for the bones of the hand or foot, without however excluding other applications, particularly in the field of the spine. Depending on the pathological case to be treated, these plates can have a general rectilinear shape or have other geometric shapes.

From this state of the art, one of the objects the invention proposes to attain is to improve, in a sure and efficient manner, the compression in a precise direction between the bone parts held by the plate.

To attain this object, an orthopedic implant has been invented and designed in the form of a plate adapted to be fixed between two bone parts by screws engaged in throughgoing holes extending through the entire thickness of the plate.

According to the invention, the formations of the plate adapted to ensure, under a screwing effect, an adjustable compression force to bring the two bone parts closer together, are formed by at least one seat having a cross-section defining several distinct regions adapted to cooperate with a screw in order to generate a progressive straight-line displacement of the screw during the screwing action corresponding to a displacement of at least one of the bone parts, causing the compression force.

To attain the given object of obtaining a controlled and adjustable compression, one of the regions is constituted of a hole having a diameter D1 adapted to receive the screw head, whereas another region is a hole having a diameter D2 less than the diameter D1, and having, over a determined height, a slope between 40° and 60°, the regions being connected by an intermediate region having a slope in the direction of the region constituted by the hole having the diameter D1.

Advantageously, the slope of the intermediate region is comprised between 15° and 30°.

Another problem that the invention proposes to resolve is to ensure a temporary fixation of the plate so as to facilitate the fixation of the latter by the operator by means of pins and to allow, after the pins are set in place in one of the bone parts, sliding under the plate at the time of screwing while ensuring a compression according to a precise direction.

To solve such a problem, the plate has, in a known manner, a guide hole for the insertion of a pin on the side of one of the bone parts, and another guide hole for the insertion of a pin on the side of the other bone part.

According to the invention, one of the guide holes is a circular hole whose diameter corresponds substantially to that of the pin, and the other guide hole is an elongated slot.

To solve the given problem of respecting the anatomy, particularly in the case of an MTP arthrodesis plate, the axis of the elongated slot extends relative to the longitudinal axis of the plate at an angle between about 1° and 10°.

Generally, the plate has smooth and/or threaded holes adapted to receive anchor screws to the bone parts.

According to this base-design of the plate and of the pathological case to be treated:
Either the different holes are aligned;
Or some of the holes are arranged according to the apex of a triangle or of a quadrilateral.

In one embodiment, the plate is longitudinally centered so as to adapt to the curvature of the bone parts.

### BRIEF DESCRIPTION OF THE DRAWINGS

The invention is described hereinafter in more detail with reference to the attached drawings, in which:

FIG. 1 is a perspective view of an embodiment of a plate according to the invention;

FIG. 2 is a partial, large-scale, perspective view showing the compression seat;

FIG. 3 is a longitudinal cross-section corresponding to FIG. 2;

FIGS. 4, 5, and 6 are partial, cross-sectional, schematic, perspective views showing the compression obtained as the screw is pressed into the compression seat;

FIG. 7 is a perspective view of another embodiment of the plate, particularly for MTP arthrodesis;

FIGS. 8, 9, and 10 are perspective views of embodiments of plates of other shapes;

FIG. 11 shows an embodiment of a plate longitudinally shaped to fit to the curvature of the bone;

FIGS. 12 to 18 are perspective views showing the installation and fixation of the plate on two bone parts for compressing same together, the bone parts being schematically shown as parallepipedal blocks;

FIG. 19 is a partial view of the plate showing, schematically, two extreme positions of the initial boring to obtain maximum compression (position 1) or no compression (position 0).

### DETAILED DESCRIPTION

The osteosynthesis and/or arthrodesis plate is indicated generally at 1. In a known manner, this plate 1 has smooth and/or threaded holes 1a for anchor screws 2 screwed into bone parts O1 and O2, as described below.

According to a characteristic of the invention, the plate 1 has at least one compression seat or formation 1b.

As shown, in particular in FIGS. 2 and 3, this seat 1b has several distinct regions 1b1, 1b2, and 1b3 that allow the desired compression to be set, particularly as a function of the bone quality and as a function of the start position of a screw 3 in the seat.

More particularly, the seat 1b is formed by three distinct regions having the following characteristics:

The region 1b1 is a hole having a diameter D1, adapted to receive the head 3a of the screw 3.

The region 1b2 is a hole having a diameter D2 less than the diameter D1 and having, over a determined height, comprised between about 1 and 2 mm, a slope on the order of 40° to 60° relative to a base plane of the plate.

The regions 1b1 and 1b2 are connected by an intermediate transition region 1b3 having a down slope, that is, angled downward in the direction of the region 1b1. This slope is between about 15° and 30°.

Considering these characteristics, compressing together the two bone parts O1 and O2 is carried out as follows, with reference, in particular, to the FIGS. 4, 5, 6, 15, and 16.

A pilot hole is first made in one of the bone parts being considered according to the region 1b2 or 1b1 as a function of the compression one wants to obtain.

The screw 3 is inserted into the hole until its head 3a contacts the plate 1 (FIGS. 4 and 5).

Since the hole of the region 1b2 has a diameter smaller than that of the screw head 3, this head immediately slides so as to be offset from the axis of the region 1b1, causing a first, rapid compression.

With continued screwing action, the screw head 3a is slides along the slope of the intermediate region 1b3 causing a second compression until it reaches a stable position fitting in the hole 1b1 whose diameter is the same as that of the screw head 3a. Naturally, it is possible to stop at any intermediate position in order to precisely adjust the compression.

It must be noted also that the extent of compression generated is on the order of 0 to 3 mm and can be controlled as a function of the position of the initial passage. Therefore, it is possible to obtain maximum compression at a seat in position 1 and no compression at a seat in a position 0 (FIG. 19).

According to another characteristic, the plate 1 has at least one guide hole 1c for the insertion of a guide pin 4 for temporarily fixing the plate 1. Advantageously, the plate 1 has a guide hole 1c for the insertion of a pin 3 on the side of one of the bone parts O1 and another guide hole 1d for the insertion of another guide pin 5 on the side of the other bone part O2.

Considering the effect of the desired compression, such as previously mentioned, the guide hole 1c is a circular hole whose diameter corresponds substantially to that of the pin 4, whereas the other guide hole 1d is an elongated slot, located on the side of the compression seat 1b.

These provisions therefore enable the bone to slide relative to the plate 1 at the time of screwing, while ensuring compression in a precise direction generally along the axis of the plate. The pins 4 and 5 are of any known and appropriate type, and perfectly know to one having ordinary skill in the art.

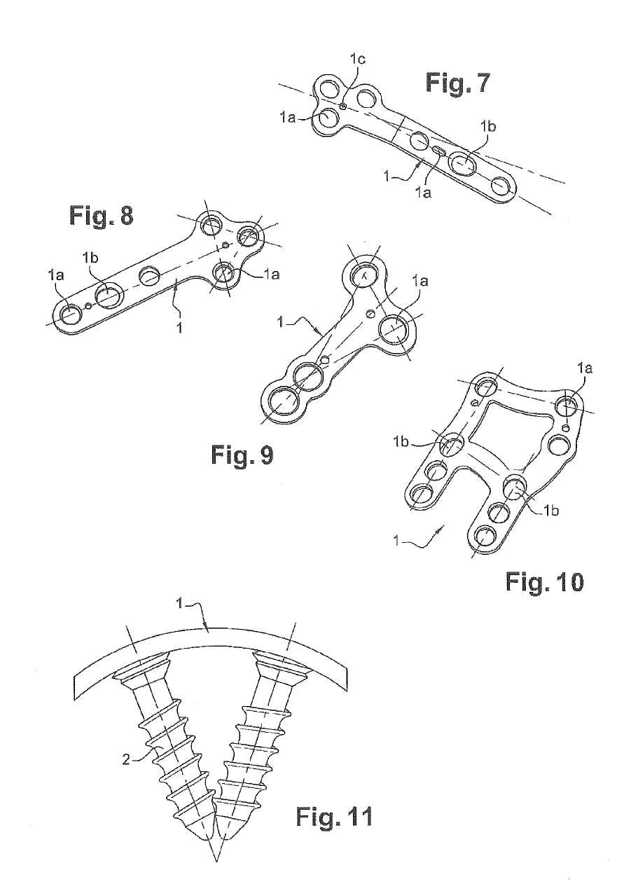

The elongated slot 1d for temporary fixation is particularly well adapted in the case of an MTP arthrodesis plate in order to take into account the anatomy according to which the angle between the phalange P1 and the metatarsus 1 is on the order of 10°. Indeed, a plate according to the state of the art makes it difficult to ensure the compression while keeping the above-described angle. According to the invention, the slot 1d only has to be angularly oriented relative to the median axis of the plate 1 according to an angle of about 5° (FIG. 7).

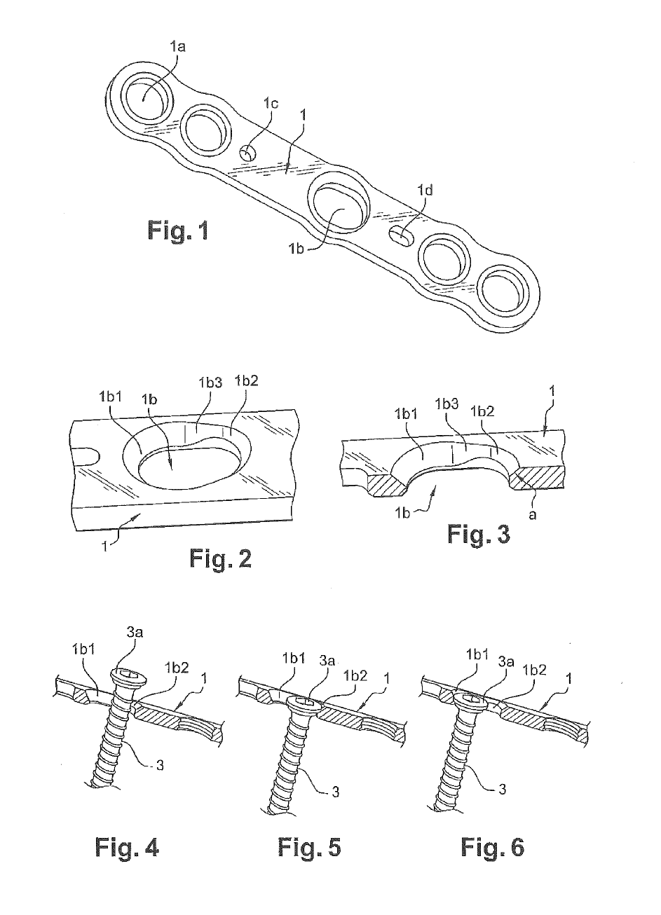

As shown in the figures of the drawings, the plate 1 can have different shapes, with the holes 1a aligned as in FIG. 1 or arranged, all or in part on the corners of a triangle or of a quadrilateral (FIGS. 8, 9, and 10). More particularly, in FIG. 8, the plate is adapted to be used in the case of a Lapidus arthrodesis, in FIG. 9, for a basal osteotomy of the first metatarsus and for a ⅔ Lisfranc arthrodesis (FIG. 10). These provisions of the screw, in a triangle or in a quadrilateral, improve the mounting stability.

It must be noted also that the plate 1, no matter its geometric shape, can be bent longitudinally so as to adapt to the curvature of the bone (FIG. 6) making it possible, as a consequence, for the screws 2 to form an angle relative to one another (FIG. 11).

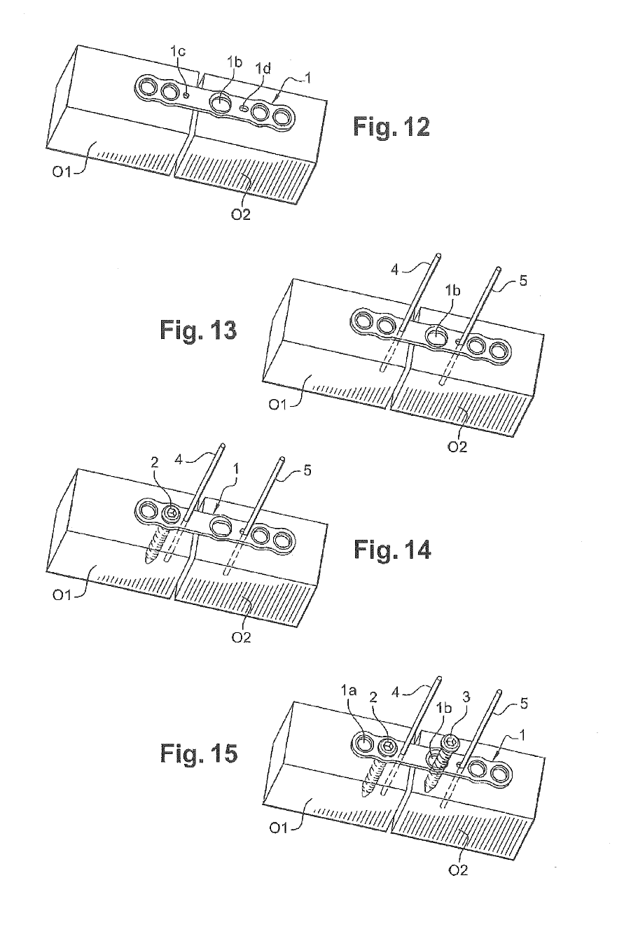

With reference to FIGS. 12 to 18 that show the setting in place of the plate according to the invention:

After the osteotomies have been carried out, the plate 1 is positioned between the two bone parts O1 and O2 (FIG. 12).

The surgeon stabilizes the plate 1 with the two temporary pins 4 and 5, one of which is engaged in the hole 1c and whereas the other one in the slot 1d (FIG. 13).

At least one screw 2 is engaged through the hole 1a and screwed into the bone part O1 where there is no compression formation 1b is not positioned (FIG. 14).

The surgeon then screws the screw 3 in the compression lug 1b, choosing the extent of compression as previously indicated (FIG. 15).

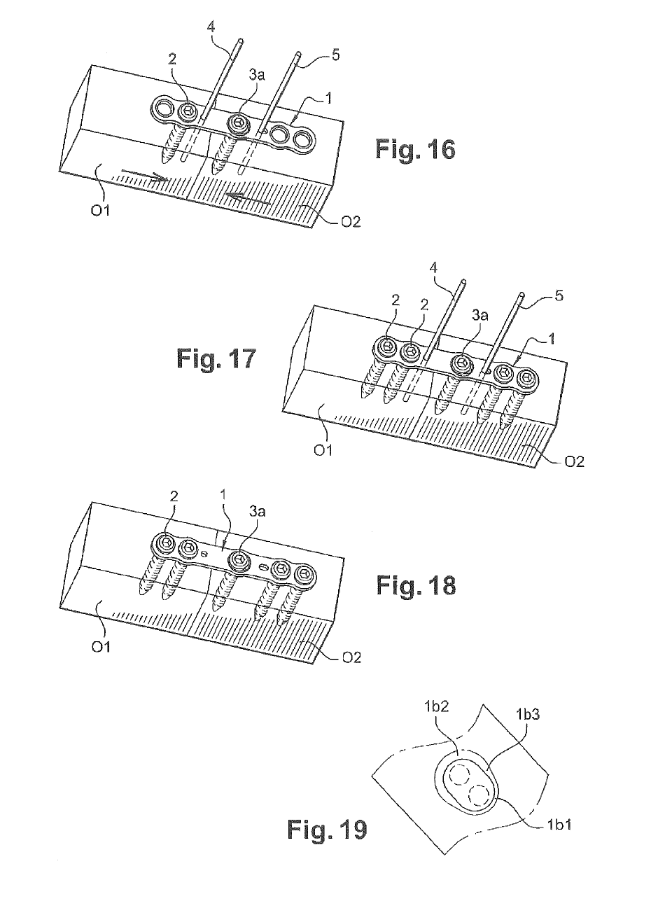

Once the compression is done (FIG. 16), the surgeon can screw in one or more additional screws 2 (FIG. 17), then remove the guide pins 4 and 5 (FIG. 18).

The advantages are readily apparent from the description.

1-8. (canceled)

9. A bone plate comprising:
first and second holes adapted to receive first and second fixation members, wherein the first hole is alignable with a first bone part and the second hole is alignable with a second bone part;
a compression slot having a surface adapted to interact with a portion of a third fixation member to cause displacement of the third fixation member when inserted into the compression slot;
a first pin hole alignable with the first bone part; and
a second pin hole alignable with the second bone part,
wherein the first pin hole has a shape that is the same as the shape of a first fixation pin receivable in the first pin hole, and the second pin hole is an elongate slot having a width that is dimensioned to closely confront a second fixation pin along a majority of the length of the second pin hole.

10. The bone plate according to claim 9, wherein the first and second holes are locking holes.

11. The bone plate according to claim 10, wherein the first and second holes include threading for engaging with a threaded section of the first and second fixation members.

12. The bone plate according to claim 9, wherein the compression slot is arranged between the first and second holes.

13. The bone plate according to claim 9, wherein the compression slot and the second pin hole each has a central axis that extends into the second but not the first bone part.

14. The bone plate according to claim 9, wherein a long axis of the second pin hole is angularly oriented relative to a median axis of the plate.

15. The bone plate according to claim 14, wherein the angle of the second pin hole relative to the median axis of the plate is within a range of about 1° to about 10°.

16. The bone plate according to claim 9, wherein the bone plate includes a plurality of holes that are arranged according to the corners of a triangle or a quadrilateral, some of the plurality of holes being alignable with the first bone part and some of the plurality of holes being alignable with the second bone part.

17. The bone plate according to claim 9, wherein a bottom surface of the bone plate is curved so as to adapt to a curvature of the first and second bone parts, the curvature of the bone plate arranging the first and second fixation members at an angle relative to one another once inserted into the first and second holes.

18. The bone plate according to claim 9, wherein the second pin hole is offset from the first pin hole in a direction extending transverse to a longitudinal axis of the plate.

19. The bone plate according to claim 9, wherein a long axis of the compression slot is substantially aligned with a long axis of the second pin hole.

20. The bone plate according to claim 9, wherein the width of the second pin hole is dimensioned to closely confront the second fixation pin along an entirety of the length of the second pin hole.

21. The bone plate according to claim 9, wherein the first pin hole is circular.

22. The bone plate according to claim 9, wherein the width of the second pin hole is approximately the same as a width of the second fixation pin.

23. A bone plate comprising a length adapted to span first and second bone parts, the bone plate having first and second pin holes alignable with the first and second bone parts, respectively, wherein the first pin hole has a shape that is the same as the shape of a first fixation pin receivable in the first pin hole, and the second pin hole is an elongate slot having a width that is dimensioned to closely confront a second fixation pin along a majority of the length of the second pin hole.

24. The bone plate according to claim 23, wherein the width of the second pin hole is dimensioned to closely confront the second fixation pin along an entirety of the length of the second pin hole.

25. The bone plate according to claim 23, further comprising:
first and second locking holes adapted to receive first and second fixation members, wherein the first locking hole is alignable with the first bone part and the second locking hole is alignable with the second bone part; and
a compression slot having a surface adapted to interact with a portion of a third fixation member to cause displacement of the third fixation member when inserted into the compression slot.

26. The bone plate according to claim 25, wherein the compression slot and the second pin hole each has a central axis that extends into the second but not the first bone part.

27. The bone plate according to claim 23, wherein the bone plate includes a plurality of holes that are arranged according to the corners of a triangle or a quadrilateral, some of the plurality of holes being alignable with the first bone part and some of the plurality of holes being alignable with the second bone part.

28. The bone plate according to claim 23, wherein a bottom surface of the bone plate is curved so as to adapt to a curvature of the first and second bone parts, the curvature of the bone plate arranging the first and second fixation members at an angle relative to one another once inserted into the first and second holes.

29. The bone plate according to claim 23, wherein the second pin hole is offset from the first pin hole in a direction extending transverse to a longitudinal axis of the plate.

30. The bone plate according to claim 23, wherein a long axis of the second pin hole is angularly oriented relative to a median axis of the plate.

31. The bone plate according to claim 23, wherein the width of the second pin hole is approximately the same as a width of the second fixation pin.

32. The bone plate according to claim 23, wherein the first pin hole is circular.

33. A bone plate comprising:
first and second locking holes adapted to receive first and second fixation members, wherein the first locking hole is alignable with a first bone part and the second locking hole is alignable with a second bone part;
a compression slot having a surface adapted to interact with a portion of a third fixation member to cause displacement of the third fixation member when inserted into the compression slot;
a first pin hole alignable with the first bone part; and
a second pin hole alignable with the second bone part,
wherein the first pin hole has a shape that is the same as the shape of a first fixation pin receivable in the first pin hole, and the second pin hole is an elongate slot having a width that is dimensioned to closely confront a second fixation pin along an entirety of the length of the second pin hole,
and wherein a long axis of the compression slot is substantially aligned with a long axis of the second pin hole, and a bottom surface of the bone plate is curved so as to adapt to a curvature of the first and second bone parts, the curvature of the bone plate arranging the first and second fixation members at an angle relative to one another once inserted into the first and second holes.
